(19) United States
(12) Patent Application Publication
(10) Pub. No.: US 2024/0207606 A1
(43) Pub. Date: Jun. 27, 2024

(54) STIMULATION INTERFACE PADS

(71) Applicant: AVATION MEDICAL, INC., Columbus, OH (US)

(72) Inventors: Manish Vaishya, Columbus, OH (US); Samantha Patrick, Columbus, OH (US); Katherine Greers, Columbus, OH (US); Nikolas Barrera, Columbus, OH (US); Maekele Gebrekidan, Columbus, OH (US); Mingming Zhang, Columbus, OH (US)

(21) Appl. No.: 18/558,202

(22) PCT Filed: May 3, 2022
(86) PCT No.: PCT/US2022/027374

§ 371 Date: Oct. 31, 2023

Related U.S. Application Data
(60) Provisional application No. 63/183,170, filed on May 3, 2021.

(51) Int. Cl. A61N 1/04

(52) U.S. Cl. CPC A61N 1/0496; A61N 1/0456

(57) ABSTRACT

A neurostimulator for applying electrical stimulation through a subjects skin includes a wearable configured to be worn by the subject and a control unit connected to the wearable. Electrodes are mounted on the wearable and electrically connected to the control unit. An interface pad overlies the electrodes and is configured to be positioned on the subjects skin. The interface pad is configured to conduct electrical signals between the electrodes and the subjects skin. The interface pad includes a body of elastomeric material supporting a conductive material. The interface pad is mounted on the wearable so that the interface pad covers the electrodes and distributes electrical current uniformly across the electrodes.

### RELATED APPLICATION

The application claims the benefit of U.S. Provisional Application Ser. No. 63/183,170, filed May 3, 2021.

### TECHNICAL FIELD

The invention relates to a wearable electronic medical device for transcutaneous electrical stimulation of peripheral nerves for the purpose of treating a subject. More specifically, the invention relates to stimulation interface pads for interfacing with the subject's skin to deliver the transcutaneous electrical stimulation.

### BACKGROUND

Current methods for peripheral nerve stimulation employ hydrogel pads to conduct electric current from stimulator electrodes to the skin. The primary functions of a hydrogel pad are to provide a seamless interface between the stimulation electrode surface and human skin, and to distribute the current flow across the surface area. Conventional hydrogel pads are often coated with adhesives to ensure a higher area of skin contact. The adhesives wear out over time, which necessitates frequent pad replacement. In addition to their limited use lifecycle, shelf life, difficulty of use, skin reactions to adhesives, and high level of difficulty to use all contribute to conventional hydrogel pads being less than ideal.

Hydrogel pads have deteriorating physical properties, either with use or with exposure to atmosphere. Because of this, hydrogel pads require frequent replacement or are used as disposables, causing usability and compliance challenges. Additionally, hydrogel pads are generally hydrophilic, which prevents proper washing or cleaning of the electrodes or a wearable carrier of the electrodes.

Accordingly, there is a clinical need for a robust, stable and long-lasting pad that provides the same performance when used with a peripheral nerve stimulation system.

### SUMMARY

This invention incorporates a novel stimulation interface pad for facilitating transcutaneous electrical neurostimulation. The interface pad has a configuration in which electrically conductive elements are embedded in a non-conductive carrier. The carrier can have a variety of configurations that implement a variety of materials and/or composites alone or in combination, with the material properties, e.g., stiffness, composition, conductivity or lack thereof, etc. The interface pads are durable, easy to use, provide superior electrical properties and transference of stimulation energy from the electrodes to the skin, with the result being a highly effective standard of care with reduced overall cost.

According to one aspect, a neurostimulator for applying electrical stimulation through a subject's skin includes a wearable configured to be worn by the subject and a control unit connected to the wearable. Electrodes are mounted on the wearable and electrically connected to the control unit. An interface pad overlies the electrodes and is configured to be positioned on the subject's skin. The interface pad is configured to conduct electrical signals between the electrodes and the subject's skin. The interface pad includes a body of elastomeric material supporting a conductive material. The interface pad is mounted on the wearable so that the interface pad covers the electrodes and distributes electrical current uniformly across the electrodes.

According to another aspect, the electrodes can be stimulation electrodes and the interface pad can be configured to deliver electrical stimulation signals from the stimulation electrodes to the subject's skin.

According to another aspect, alone or in combination with any other aspect, the electrodes can be recording electrodes and the interface pad can be configured to deliver EMG response signals from the subject's skin to the recording electrodes.

According to another aspect, alone or in combination with any other aspect, the neurostimulator can also include a structure for securing the interface pad to the wearable. The structure can be configured to urge the interface pad into engagement with the electrodes so that the body of elastic material deforms elastically onto the electrodes to form a seal that seals the electrodes behind the body of elastomeric material.

According to another aspect, alone or in combination with any other aspect, the structure can be configured to urge the interface pad uniformly against the electrodes in so that the interface pad exhibits uniform conductivity across the electrodes.

According to another aspect, alone or in combination with any other aspect, the structure can include a snap-fit structure including an electrode-associated component secured to the wearable and a interface pad-associated component that engages and supports the body of elastomeric material. The interface pad-associated component can be connectable to the electrode-associated component via a snap-fit.

According to another aspect, alone or in combination with any other aspect, the electrode-associated component and the interface pad-associated component can be configured so that the interface pad is urged against and deformed into engagement with the electrodes when the snap-fit connection is established.

According to another aspect, alone or in combination with any other aspect, the body of elastomeric material can be a body of silicone material.

According to another aspect, alone or in combination with any other aspect, the conductive material can be one or more of the following materials: carbon, carbon fibers, graphite, carbon nanotubes, copper (Cu), nickel (Ni), silver (Ag), aluminum (Al), Ag/Cu alloy, Ni/Al alloy, AG/Al alloy, Ag/Ni alloy, nickel coated graphite, silver coated glass, silver plated aluminum, silver coated fabric, conductive spray coating, conductive foam, a silver fabric coated silicone sponge, a silver fabric lined silver plated silicone.

According to another aspect, alone or in combination with any other aspect, the body of elastomeric material can be a single layer sheet of silicone and the conductive material can be conductive particles embedded in the sheet of silicone. The particles can be configured so that an impedance across an area of the interface pad is uniform. The uniformity of the impedance can be determined through the type of conductive material, the density of the conductive material in the body of elastomeric material, the orientation of filler material particles in the body of elastomeric material, or a combination thereof.

According to another aspect, alone or in combination with any other aspect, the interface pad can have a multi-layered construction including at least one low conductivity layer and at least one high conductivity layer.

According to another aspect, alone or in combination with any other aspect, the conductivity of the layers can be determined by the type of filler material in each layer, the amount or density of filler material in each layer, the orientation of filler material particles in each layer, or a combination thereof.

According to another aspect, alone or in combination with any other aspect, the interface pad can include a high conductivity layer sandwiched in between two low conductivity layers. The high conductivity layer can be configured to act as a current spreader that helps spread electrical current passing into the high conductivity layer through one or more localized regions of a first of the low conductivity layers so that the current is spread evenly across the area of a second of the low conductivity layers.

According to another aspect, alone or in combination with any other aspect, the first low conductivity layer can be configured to interface with the electrodes and the second low conductivity layer can be configured to interface with the subject's skin. The interface pad can be configured to deliver stimulation current to the subject's skin uniformly across the interface pad from localized areas where the electrodes interface the first layer.

According to another aspect, alone or in combination with any other aspect, the first low conductivity layer can be configured to interface with the subject's skin and the second low conductivity layer can be configured to interface with the electrodes the interface pad being configured to distribute localized EMG response current from the subject's skin uniformly across the interface pad to the electrodes interfacing the second layer.

According to another aspect, alone or in combination with any other aspect, the conductive material can include conductive particles embedded in the body of elastomeric material. The particles can be oriented with a bias to produce at least one of a predetermined conductive pathway and a predetermined non-isotropic conductivity.

According to another aspect, alone or in combination with any other aspect, the conductive particles embedded in the body of elastomeric material can be biased to an orientation at a predetermined angle relative to the thickness of the body of elastomeric material.

According to another aspect, alone or in combination with any other aspect, the interface pad can include multiple layers of elastomeric material, each having conductive particles biased to a predetermined orientation configured so that the interface pad as a whole displays predetermined conductivity and/or impedance characteristics.

According to another aspect, alone or in combination with any other aspect, the interface pad can include multiple elastomeric layers with conductive particles embedded therein. The multiple layered structure can be configured to provide higher interstitial capacitance in order to block DC current in the interface pad.

According to another aspect, alone or in combination with any other aspect, the interface pad can include one or more elastomeric layers with conductive particles embedded therein. The interface pad can also include an embedded dry electrolyte in one or more of the elastomeric layers. The dry electrolyte can be configured to provide a desired ionization response.

### DRAWINGS

FIG. 1 is a schematic illustration depicting a neurostimulation system, according to an example configuration.

FIG. 2 is a schematic illustration depicting an example implementation of the neurostimulation system.

FIG. 3 is a schematic illustration depicting a stimulation interface pad portion of the system, according to an example configuration.

FIG. 4 is a cross-sectional representation of a portion of the neurostimulation system, according to an example configuration.

FIG. 5 is a cross-sectional representation of the interface pad, according to an example configuration.

FIG. 6 are cross-sectional representations of portions of the interface pad, according to another example configuration.

### DESCRIPTION

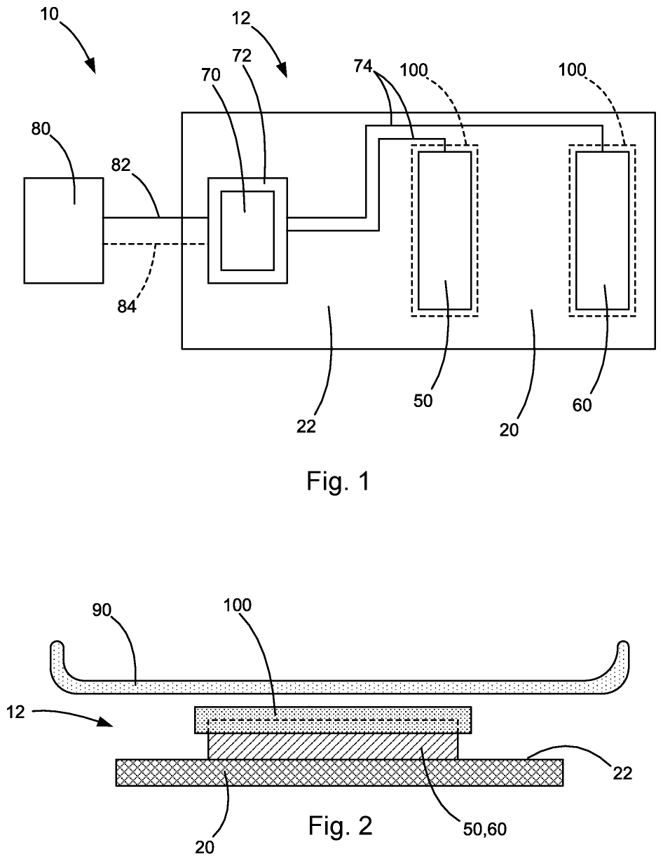

FIG. 1 illustrates schematically a neurostimulation system 10 including a wearable neurostimulator 12 that can implement the reusable interface pads disclosed herein. The configurations of the neurostimulation system 10 and the neurostimulator 12 shown in FIG. 1 are by way of example only and are by no means limiting. For instance, the stimulation system 10 can be similar or identical to those described in U.S. Pat. No. 11,141,586 B2, the disclosure of which is hereby incorporated by reference in its entirety.

The neurostimulator 12 includes a wearable 20, such as a strap, brace, sleeve, etc. configured to support various components adapted to deliver neurostimulation to a subject. The form factor of the wearable 20 is not important, as the reusable interface pads disclosed herein are agnostic to the position/orientation of the neurostimulator 12 on the subject and the body part of the subject to which the neurostimulation is applied.

The neurostimulator 12 includes two or more stimulation electrodes 50, e.g., an electrode array, that are arranged on an inner surface 22 of the wearable 20 that is configured to face the subject's skin when in use. The number of stimulation electrodes 50, the area covered by the stimulation electrodes, the stimulation electrode density (i.e., number of stimulation electrodes per unit area), and the distribution, grouping, or pattern of stimulation electrodes all can vary depending on the intended application of the neurostimulator 12. The stimulation electrodes 50 are configured to apply transcutaneous electrical neurostimulation to the subject.

The neurostimulator 12 can also include recording electrodes 60 arranged on the inner surface 22 of the wearable 20 spaced from the stimulation electrodes 50. The number of recording electrodes 60, the area covered by the recording electrodes, the recording electrode density (i.e., number of recording electrodes per unit area), and the distribution, grouping, or pattern of recording electrodes all can vary depending on the intended application of the neurostimulator 12. The recording electrodes 60 are configured to record physiological responses from the subject (e.g., neurological, muscular, neuromuscular, etc.).

The responses recorded via the recording electrodes 60 can be those elicited by the neurostimulation applied via the stimulation electrodes 50. This can, for example, facilitate the utilization of responses to stimulation sensed by the recording electrodes 60 as feedback in a closed-loop stimulation control scheme. The spacing between the stimulation electrodes 50 and the recording electrodes 60 can be important, as it can be necessary to provide adequate distance between the electrodes so that electrical stimulation signals can be separated or distinguished from the elicited responses.

The neurostimulator 12 also includes an electronic control unit 70 that is operative to control the application of transcutaneous electrical nerve stimulation via the stimulating electrodes 50 and to receive stimulation feedback gathered by the recording electrodes 60. The control unit 70 is located on an outer surface of the wearable 20, opposite the inner surface 22, and is therefore shown in dashed lines. The control unit 70 can be is detachably connected to the remainder of the neurostimulator 12 via a plug-in or snap-in connector 72, which allows the control unit to be utilized with other neurostimulator configurations and also allows the wearable 20 and the components remaining on the wearable (e.g., the electrodes, etc.) to be replaced when worn out, expired, or otherwise due for replacement.

The connector 72 can support a plurality of terminals for electrically connecting the control unit 70 to the stimulation electrodes 50 and the recording electrodes 60. Certain terminals in the connector 72 can be electrically connected to the stimulation electrodes 50 by wires or leads 74 that are mounted, embedded, or otherwise connected to the wearable 20. Through these connections, the control unit 70 can control the application of stimulation energy via the stimulation electrodes 50 and can monitor elicited responses via the recording electrodes 60.

The control unit 70 is configured to communicate via wired and/or wireless connection to an external device 80, such as a computer, tablet, smartphone, or a custom programming device. For example, in a user mode, the external device 80 can be a smartphone running a customized application that enables the app enables a user to control the control unit 70 to apply stimulation in a self-applied stimulation mode. As another example, in a programming or physician's mode, the prescribing party that oversees the neurostimulation can use an external device 80 in the form of a computer (e.g., PC/MAC), tablet, or other device running customized software that communicates with the control unit 70 to allow for device setup, calibration, customization, downloading recorded data, uploading stimulation parameters, etc.

The stimulation electrodes 50 and recording electrodes 60 can be dry electrodes, which require the use of a interface pad 100 to interface with the subject's skin, i.e., to deliver stimulation energy via the skin and to monitor elicited responses through the subject's skin. The interface pad 100 can be shaped and sized to coincide with and cover the stimulation electrodes 50. In use, the interface pad 100 facilitates a strong, reliable electrical connection between the stimulation electrodes 50 and the subject's skin.

The interface pad 100 is configured to overcome the drawbacks associated with conventional hydrogel pads. An example implementation of the interface pad 100 is shown in FIG. 2. Referring to FIG. 2, the interface pad 100 serves as the electrically conductive medium between the subject's skin 90 and the stimulation electrodes 50. Similarly, the interface pad 100 can also serve as the electrically conductive medium between the recording electrodes 60 and the subject's skin 90.

As shown in FIGS. 1 and 2, the neurostimulator 12 is fit with the interface pad 100, which at least partially covers the stimulation electrodes 50 and/or the recording electrodes 60. The interface pads 100 of FIG. 1 are illustrated in hidden lines (dashed) in FIG. 1 so that the configuration of the neurostimulator 12 remains clear. The interface pads 100 are fixed to the electrodes 50, 60 and/or the wearable device 20. In fact, according to one example configuration, the electrodes 50, 60 can be permanently fixed or embedded within the interface pad 100, as shown in dashed lines in FIG. 2.

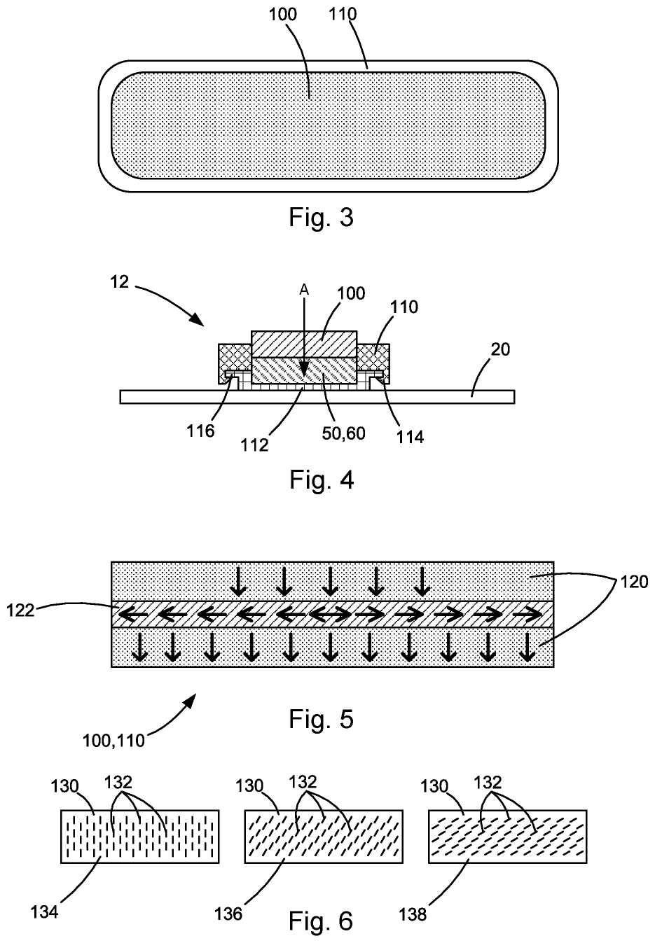

Referring to FIG. 3, the interface pad 100 can be supported on a base or substrate 110 that facilitates its connection to the neurostimulator 12, i.e., to/on the wearable 20 and/or the electrodes 50, 60. The base 110 can, for example, be a housing (e.g., plastic) that connects mechanically to the neurostimulator 12, placing the interface pad 100 in direct contact with the electrodes 50, 60. The connection can, for example, be provided by structure that produces a snap-fit connection that is releasable so that the interface pads 100 can be washed, swapped, or replaced.

In one particular configuration illustrated in FIG. 4, the snap-fit can be facilitated by structure in the form of a mating component mounted to the wearable 20 adjacent the electrodes 50, 60 or even configured as a portion (e.g., base portion) of the electrodes. The structure producing this snap-fit can include a component associated with the electrodes 50, 60 and a component associated with the interface pad 100. In the example of FIG. 4, one component of the snap-fit structure can be a bezel or housing 112 that supports the electrodes 50, 60, respectively, on the wearable 20. In this configuration, the other component of the snap-fit structure can be snap hooks 114 on the base 110 that are configured to engage a corresponding lip 116 on the housing 112.

According to the snap-fit structure of FIG. 4, the connection of the base 110 to the wearable 20/electrodes 50, 60 can be configured so that the interface pad 100 is pressed firmly against the electrodes 50, 60. In fact, the base 110 and the housing 112 can be configured to form an interference between the interface pad 100 and the electrodes 50, 60 so that the interface pad is deformed into engagement with the electrodes. In this manner, the resilience of the elastomeric material used to construct the interface pad 100 can maintain an effective contact between the pad and the electrodes 50, 60.

The interface pad 100 has an electrically conductive construction. According to one example construction, the interface pad 100 includes an elastomeric material impregnated or embedded with an electrically conductive material. Various elastomers, such as thermoplastic elastomers and rubber, can be implemented. Because it is a commonly used material both for skin and tissue contact in the medical device field, silicone can be an ideal material for constructing the interface pad 100. In this example construction, silicone is embedded with one or more electrically conductive fillers. While silicone provides a soft, comfortable and flexible skin contact, the filler provides the required electrical conductivity for conducting the electrical stimulation signals from the stimulation electrodes 50 to the skin 90, and from the skin to the recording electrodes 60. The conductive properties of the interface pad 100 can be modified through the selection of the conductive filler material and/or the density of the conductive filler material in the pad.

The electrically conductive materials used to form the filler can vary. The electrically conductive filler can, for example, include carbon-based fillers, such as carbon, carbon fibers, graphite, or carbon nanotubes. The filler can also include metals, such as copper (Cu), nickel (Ni), silver (Ag), or aluminum (Al). Metal alloys, such as Ag/Cu alloys, Ni/Al alloys, AG/Al alloys, or Ag/Ni alloys can also be used as a conductive filler. Additionally, material combinations such as nickel coated graphite, silver coated glass, silver plated aluminum, silver coated fabric, conductive spray coated silicone, conductive foam, silver fabric coated silicone sponge, and silver fabric lined silver plated silicone can also be used.

A silicone material construction of the interface pad 100 is both durable and washable, as is the wearable device 20. The electrodes 50, 60 can be shielded from moisture by the interface pad 100, which covers the electrodes. The aforementioned mechanical connection of the interface pads 100 to the electrodes/wearable, and the resulting compression of the pads onto the electrodes 50, 60 can help bolster this shielding. The interface pads 100 being installed on a neurostimulator 12 on a wearable 20 allows for skin placement without the need for adhesives, which deteriorate over time. The interface pad 100, being free from adhesives, is also washable and can be used repeatedly without deteriorating. The neurostimulator 12 can therefore significantly reduce or eliminate the burden of pad replacement. At the same time, the interface pads 100 can maintain a high electrical conductivity contact with the skin. Additionally, because the silicone pad shields the electrode, it also substantially reduces the risk of localized skin burn, in the event a patient improperly applies or fails to use the gel pads.

The interface pad 100 can be manufactured in a variety of manners, implementing the different filler materials and/or different elastomeric materials in various combinations. The interface pads 100 can be manufactured by molding a sheet of the elastomeric material with embedded conductive fillers and cutting or stamping the pads from the sheet using, for example, a cutting die. Other manufacturing methods, such as injection molding or compression molding, can also be used to manufacture the interface pads 100. The interface pads 100 can also be fixed to the neurostimulator, i.e., to the wearable device 20 and/or the electrodes 50, 60 in a variety of manners in addition to the snap-fit structure described with reference to FIG. 4. These additional fixations can include, for example, adhesives, mechanical fasteners, thermal bonding, stitching, or co-building the wearable and pad by way of an overmolding process.

An interface between the skin 90 and stimulation electrodes 50 has certain technical performance characteristics that can be measured and used to determine the effectiveness of the neurostimulator 12. The interface pads 100 achieves and enhances these characteristics in a design implementing a long-lasting conductive elastomeric material construction. Among the advantageous technical performance characteristics realized through this construction are:
Uniform distribution of current.
Reduced current density.
Efficient conduction of current from electrode to skin.
Finite impedance and DC block for stimulation current.
Enhanced impedance control.

From the above, it will be appreciated that the interface pad can be constructed in various configurations that combine the aforementioned features, materials, and constructions in different combinations to achieve these technical performance characteristics.

### Example Configuration 1

According to one example configuration, the interface pad 100 can be a single layer sheet of silicone impregnated with conductive particles or having conductive particles embedded therein. With its single layer silicone construction, the interface pad can provide a uniform pressure distribution with the skin, and hence a controlled and low impedance determined by the conductive filler particles. The interface pad 100 can thus provide a controlled, finite impedance, which produces an efficient conduction of current from the electrode to the skin (and vice versa), with a uniform current distribution and reduced current density. The impedance can be determined in a variety of manners, such as through the type of filler material, the amount or density of filler material, the orientation of filler material particles in the pad or a combination thereof.

### Example Configuration 2

Referring to FIG. 5, according to another example configuration, the interface pad 100 can be a multi-layered pad including at least one low conductivity layer 120 and at least one high conductivity layer 122. In the example configuration of FIG. 5, the interface pad 100 includes two low conductivity layers 120 and two high conductivity layers 122. The conductivity of the layers can be determined, for example, by the type of filler material in each layer, the amount or density of filler material in each layer, the orientation of filler material particles in each layer, or a combination thereof.

As an illustration, for example, the conductivity of low conductivity layers 120 can be small, e.g., 1/10, of the conductivity of the high conductivity layer 122. In this case, even if the current inflow was not uniform, the high conductivity of layer 122 spreads the current by allowing it to flow transversely due to its high conductivity, which promotes the spread and flow of electrical current throughout the layer. The evenly distributed current can then flow uniformly distributed through the remaining layers.

FIG. 5 illustrates current flow through various layers 120, 122 of the interface pad 100 with arrows. In FIG. 5, the current flowing through the upper low conductivity layer 120 is concentrated toward the center, as indicated by the current arrows being located generally centrally in that layer. The middle, high conductivity layer 122 spreads and distributes more evenly the current from the top layer 120. As a result, the current is transferred from the middle layer 122 to the bottom low conductivity layer 120 evenly, and the current passes through the bottom layer as such. As a result, the multi-layer configuration of the interface pad 100 produces a uniform current flow so that the stimulation can be applied, as intended, to the desired area on the subject's skin 90 via the stimulation electrodes 50. Similarly, the layers of the interface pad 100 of FIG. 5 can spread/distribute the current collected from the desired location on the skin 90, which is sensed by the recording electrodes. The interface pad 100 can thus provide a controlled, finite impedance, which produces an efficient conduction of current from the electrode to the skin (and vice versa), with a uniform current distribution and reduced current density.

### Example Configuration 3

According to another example configuration, the interface pad 100 can be constructed with one or more elastomeric layers with conductive particles embedded therein. The conductive particles can be oriented with a bias to achieve this preferential pathway and non-isotropic conductivity. FIG. 6 illustrates three different layers that can be implemented in the interface pad 100. Each layer is a body of elastomeric material 130 as disclosed herein, with conductive particles 132, represented by lines, embedded therein.

A first layer or body 134 has particles 132 arranged vertically, with that vertical representation being indicative of the direction or orientation of the conductive particles. This vertical direction is transverse to the thickness of the layer 134 and thus indicates that the layer is highly conductive, at least comparatively speaking. A second layer or body 136 has particles 132 arranged at an angle representing the direction or orientation of the conductive particles. This direction, being angled and non-vertical relative to the thickness of the layer 136, indicates that the layer is less conductive than the highly conductive layer 134, comparatively speaking. A third layer or body 138 has particles 132 arranged at an angle representing the direction or orientation of the conductive particles. The angle of the particles 132 of layer 138 is greater (with respect to vertical) than the angle of the particles of layer 136. This indicates that the layer is less conductive than both the first layer 134 and the second layer 136, again comparatively speaking.

Accordingly, it will be appreciated that the conductivity of the interface pad 100 can be tailored through the arrangement of layers with conductive particles arranged at different orientations. The interface pad 100 can therefore be configured to focus its sensitivity in certain areas. Additionally, the orientation of conductive particles in this example configuration can be combined with the variable conductivity layers of example configuration 2 (see above), for example, so that the current is distributed evenly through example configuration 2, and then focused according to example configuration 3, so that the focused current is uniforms across the focused areas. The interface pad 100 can thus provide a controlled, finite impedance, which produces an efficient conduction of current from the electrode to the skin (and vice versa), with a uniform current distribution and reduced current density.

### Example Configuration 4

According to another example configuration, the interface pad 100 can be constructed with multiple elastomeric layers with conductive particles embedded therein. The layers can, for example, be arranged as described herein in regard to other example configurations. The multiple layered structure may provide higher interstitial capacitance, thus block DC current in a manner similar to that achieved by hydrogel materials. At the same time, the interface pad 100 can also provide a controlled, finite impedance, which produces an efficient conduction of current from the electrode to the skin (and vice versa), with a uniform current distribution and reduced current density.

### Example Configuration 5

According to another example configuration, the interface pad 100 can be constructed with one or more elastomeric layers with conductive particles embedded therein. Additionally, the one or more layers can also include an embedded dry electrolyte in order to provide ionization response similar to those provided by a hydrogel with saline. At the same time, the interface pad 100 can also provide a controlled, finite impedance, which produces an efficient conduction of current from the electrode to the skin (and vice versa), with a uniform current distribution and reduced current density.

### Example Embodiment 6

According to another example configuration, the interface pad 100 can be constructed with one or more elastomeric layers with conductive particles embedded therein. The one or more layers can be constructed with a very low hardness elastomeric substrate material. For example, the substrate material can be a silicone material with a hardness ranging from 30 to 70 Shore A hardness. Combined with adequate mounting pressure, this soft substrate material can eliminate localized low-resistivity spots and provide maximum comfort. The mounting pressure can be provided by the base 110 and housing 112 mounting described above in reference to FIG. 4. This overcomes the limitation of hydrogels which, after partial drying, can cause localized current paths. At the same time, the interface pad 100 can also provide a controlled, finite impedance, which produces an efficient conduction of current from the electrode to the skin (and vice versa), with a uniform current distribution and reduced current density.

Sample configurations were tested for electrical properties and the resistance values are listed below. Each sample consisted of a single layer pad, with the resistance being measured across opposing faces of the pad. Each sample was used in conjunction with a wearable garment with embedded electrodes, while ensuring that the surface of the electrode was generally covered by the interface pads 100. There was no direct contact between the skin and the electrodes.

| Pad Material/Embedded Material | Resistance (ohms) |
|---|---|
| Silver Plated Aluminum | 12.7 |
| Silver Coated Fabric | 0.7 |
| Conductive Spray Coated Silicone | 31k |
| Conductive Foam | 1.8 |
| Silver Fabric Coated Silicone Sponge | 1.2 |
| Silver Fabric Lined Silver Plated Silicone | 152 |

While the resistance values vary among these materials and constructions, each of these samples was able to provide and transmit stimulation current without noticeable discomfort. Furthermore, each sample was able to elicit a muscular response, validating that the nerve was successfully recruited and was able to be stimulated.

From this, it can be appreciated that, the electrical performance of the interface pad 100 can be tailored through the careful selection of material properties of its components. For example, the number of layers, the proportion of axial vs. transverse resistivity, the surface properties of each layer, the relative conductivity of each layer, and the form factor of the interface pad itself can be adjusted and/or combined in order to arrive at interface pad configurations with a desired electrical performance characteristics, such as resistance and capacitance.

While aspects of this disclosure have been particularly shown and described with reference to the figures and the examples described above, it will be understood by those of ordinary skill in the art that various additional aspects may be contemplated. For example, in this description, the electrical performance of the interface pad is described in terms of conductivity and in terms of impedance. These properties are, of course, inverse in that as impedance increases, conductivity decreases, and vice versa. It will therefore be appreciated that performance of the interface pad described with reference to one of these properties can also be considered as describing the pad with reference to the other of the properties. Additionally, a device or method incorporating any of the features described herein should be understood to fall under the scope of this disclosure as determined based upon the claims below and any equivalents thereof. Other aspects, objects, and advantages can be obtained from a study of the drawings, the disclosure, and the appended claims.

We claim:

1. A neurostimulator for applying electrical stimulation through a subject's skin comprising:
a wearable configured to be worn by the subject;
a control unit connected to the wearable;
electrodes mounted on the wearable and electrically connected to the control unit;
an interface pad overlying the electrodes, wherein the interface pad is configured to be positioned on the subject's skin, the interface pad being configured to conduct electrical signals between the electrodes and the subject's skin, the interface pad comprising a body of elastomeric material supporting a conductive material; and
wherein the interface pad is mounted on the wearable so that the interface pad covers the electrodes, wherein the interface pad is configured to distribute electrical current uniformly across the electrodes.

2. The neurostimulator recited in claim 1, wherein the electrodes comprise stimulation electrodes and the interface pad is configured to deliver electrical stimulation signals from the stimulation electrodes to the subject's skin.

3. The neurostimulator recited in claim 1, wherein the electrodes comprise recording electrodes and the interface pad is configured to deliver EMG response signals from the subject's skin to the recording electrodes.

4. The neurostimulator recited in claim 1, further comprising a structure for securing the interface pad to the wearable, the structure being configured to urge the interface pad into engagement with the electrodes so that the body of elastic material deforms elastically onto the electrodes to form a seal that seals the electrodes behind the body of elastomeric material.

5. The neurostimulator recited in claim 4, wherein the structure is configured to urge the interface pad uniformly against the electrodes in so that the interface pad exhibits uniform conductivity across the electrodes.

6. The neurostimulator recited in claim 4, wherein the structure comprises a snap-fit structure including an electrode-associated component secured to the wearable and a interface pad-associated component that engages and supports the body of elastomeric material, wherein the interface pad-associated component is connectable to the electrode-associated component via a snap-fit.

7. The neurostimulator recited in claim 6, wherein the electrode-associated component and the interface pad-associated component are configured so that the interface pad is urged against and deformed into engagement with the electrodes when the snap-fit connection is established.

8. The neurostimulator recited in claim 1, wherein the body of elastomeric material comprises a body of silicone material.

9. The neurostimulator recited in claim 1, wherein the conductive material comprises one or more of the following materials: carbon, carbon fibers, graphite, carbon nanotubes, copper (Cu), nickel (Ni), silver (Ag), aluminum (Al), Ag/Cu alloy, Ni/Al alloy, AG/Al alloy, Ag/Ni alloy, nickel coated graphite, silver coated glass, silver plated aluminum, silver coated fabric, conductive spray coating, conductive foam, a silver fabric coated silicone sponge, a silver fabric lined silver plated silicone.

10. The neurostimulator recited in claim 1, wherein the body of elastomeric material comprises a single layer sheet of silicone and the conductive material comprises conductive particles embedded in the sheet of silicone, wherein the particles are configured so that an impedance across an area of the interface pad is uniform, the uniformity of the impedance being determined through the type of conductive material, the density of the conductive material in the body of elastomeric material, the orientation of filler material particles in the body of elastomeric material, or a combination thereof.

11. The neurostimulator recited in claim 1, wherein the interface pad can have a multi-layered construction including at least one low conductivity layer and at least one high conductivity layer.

12. The neurostimulator recited in claim 11, wherein the conductivity of the layers can be determined by the type of filler material in each layer, the amount or density of filler material in each layer, the orientation of filler material particles in each layer, or a combination thereof.

13. The neurostimulator recited in claim 11, wherein the interface pad comprises a high conductivity layer sandwiched in between two low conductivity layers, the high conductivity layer being configured to act as a current spreader that helps spread electrical current passing into the high conductivity layer through one or more localized regions of a first of the low conductivity layers so that the current is spread evenly across the area of a second of the low conductivity layers.

14. The neurostimulator recited in claim 13, wherein the first low conductivity layer is configured to interface with the electrodes and the second low conductivity layer is configured to interface with the subject's skin, the interface pad being configured to deliver stimulation current to the subject's skin uniformly across the interface pad from localized areas where the electrodes interface the first layer.

15. The neurostimulator recited in claim 13, wherein the first low conductivity layer is configured to interface with the subject's skin and the second low conductivity layer is configured to interface with the electrodes the interface pad being configured to distribute localized EMG response current from the subject's skin uniformly across the interface pad to the electrodes interfacing the second layer.

16. The neurostimulator recited in claim 1, wherein the conductive material comprises conductive particles embedded in the body of elastomeric material, the particles being oriented with a bias to produce at least one of a predetermined conductive pathway and a predetermined non-isotropic conductivity.

17. The neurostimulator recited in claim 16, wherein the conductive particles embedded in the body of elastomeric material are biased to an orientation at a predetermined angle relative to the thickness of the body of elastomeric material.

18. The neurostimulator recited in claim 17, wherein the interface pad comprises multiple layers of elastomeric material, each having conductive particles biased to a predetermined orientation configured so that the interface pad as a whole displays predetermined conductivity and/or impedance characteristics.

19. The neurostimulator recited in claim 1, wherein the interface pad comprises multiple elastomeric layers with conductive particles embedded therein, the multiple layered structure being configured to provide higher interstitial capacitance in order to block DC current in the interface pad.

20. The neurostimulator recited in claim 1, wherein the interface pad comprises one or more elastomeric layers with conductive particles embedded therein, the interface pad further comprising an embedded dry electrolyte in one or more of the elastomeric layers, the dry electrolyte being configured to provide a desired ionization response.
